(19) United States
(12) Patent Application Publication
(10) Pub. No.: US 2022/0370246 A1
(43) Pub. Date: Nov. 24, 2022

(54) APPARATUS AND METHOD FOR PHACOEMULSIFICATION

(71) Applicant: ART, LIMITED, Grand Cayman (KY)

(72) Inventors: Takayuki Akahoshi, Tokyo (JP); Ravi Nallakrishnan, Willowbrook, IL (US)

(21) Appl. No.: 17/642,822

(22) PCT Filed: Oct. 24, 2019
(86) PCT No.: PCT/US19/57764

§ 371 Date: Mar. 14, 2022

(51) Int. Cl. A61F 9/007

(52) U.S. Cl. CPC A61F 9/00745

(57) ABSTRACT

A phacoemulsification needle is provided for emulsifying body tissue. The needle is adapted to be attached to a phacoemulsification handpiece that imparts a vibration to the needle. The needle has a hollow body with an operative distal end, a proximal end for being attached to a handpiece, and an internal surface defining an aspiration passage extending between the proximal and distal ends. In one form, the needle body includes a tip at the distal end and the needle body, the tip including one or more lateral apertures for enhanced posterior capsule safety. In another form, the needle body includes a tip including at least a pair of offset internal concave surfaces for enhanced efficiency.

### FIELD OF THE INVENTION

This disclosure relates to surgical instruments used in surgery and methods of use thereof, and more particularly to phacoemulsification apparatuses and methods of use for ophthalmic surgery.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of about 12 mm in length.

Later techniques focused on removing diseased lenses and inserting replacement artificial lenses through as small an incision as possible, about 5 mm in length. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece attached to a proximal end of a hollow needle. In the handpiece, an electrical energy is applied to a piezoelectric crystal to vibrate the distal, working end of the needle at ultrasonic frequencies in order to fragment the diseased lens into small enough particles to be aspirated from the eye through an aspiration passage in the hollow needle. Commonly, an infusion sleeve is mounted around the needle at the distal end to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage may be permanent because these cells do not regenerate. Some benefits of using as small an incision as possible during such surgery are the minimization of leakage of liquid during and after surgery to help prevent tissue collapse, faster healing time, and decreased post-operative astigmatism.

Many phacoemulsification needles and tips are designed for use with handpieces that vibrate the needle longitudinally at relatively low frequencies. In addition to longitudinal vibration, certain handpieces impart a torsional motion to the needle at an oscillation frequency of about 100 cycles per second. There are also handpieces that provide torsional oscillation of the phacoemulsification tip at frequencies of about 32,000 cycles per second. Alternatively, some handpieces, such as the Cetus ARC Nano laser, utilize laser pulses with no moving mechanical parts to emulsify the nucleus of the eye.

Use of the torsional-type handpiece has called for phacoemulsification needle tip designs differing from those used with the longitudinal-type handpiece. For example, needles have been designed with tips that are shaped, swaged and angled to take advantage of the needle motion created by the handpiece.

There are known phacoemulsification systems, such as the Centurion® System manufactured by Alcon Laboratories of Ft. Worth, Tex., which allow the surgeon to choose between using torsional motion, longitudinal motion, or a blend thereof with a single handpiece. Other common systems include the Sovereign® System, Whitestar Signature® System, Signature Ellips® FX System manufactured by Johnson & Johnson of Santa Ana, Calif. and the Stellaris® System manufactured by Bausch & Lomb of Rochester, N.Y. Common frequencies for longitudinal oscillation range from 29 Hz to 43 Hz. Common frequencies for torsional oscillation range from 31 Hz to 38 Hz. A common blended setting uses torsional motion two-thirds of the time, and longitudinal motion one-third of the time. It is believed that the “blended” motion produces a more three-dimensional effect because of the back-and-forth motion imparted during longitudinal phacoemulsification and the eccentric motion produced at the tip during torsional phacoemulsification.

Many surgeons favor phacoemulsification needles having the straight tip design commonly used with longitudinal handpieces. The great majority of surgeons use longitudinal handpieces rather than the torsional handpieces, often because torsional phacoemulsification equipment is more expensive than longitudinal equipment, and thus these surgeons find themselves unable to take advantage of the enhanced phacoemulsification results claimed by the torsional phacoemulsification systems.

With reference to U.S. Pat. Nos. 8,764,782 and 8,992,459, which are incorporated by reference herein in their entireties, the inventors have previously found that forming a phacoemulsification needle having a tip in an off-axis position relative to the axis of the aspiration passage extending through the needle body causes an eccentric motion or “wobble” during torsional phacoemulsification and improves the efficiency of phacoemulsification. Surprisingly, the inventors have also found that forming the tip in such an off-axis position also increases the efficiency of phacoemulsification when using a longitudinal handpiece. Preliminary clinical examinations indicate that using an off-axis needle with a longitudinal handpiece may be more efficient than using the same needle with a torsional hand piece providing 100% torsional action, where efficiency is measured by the energy dissipated during phacoemulsification. When used herein, the term “dissipated energy” refers to the amount of energy, most commonly measured in joules, used by the handpiece during phacoemulsification. Lower dissipated energy readings mean that less heat is being produced during phacoemulsification, which in turn lowers the possibility of thermal damage to the delicate eye tissues.

Use of an off-axis tip with a longitudinal hand piece appears to create a hybrid type of phacoemulsification motion without using the more complex and expensive torsional phacoemulsification apparatus. The inventors have also determined that the eccentric or wobble type of motion can be imparted to a phacoemulsification needle with no flare at the tip by forming the central aspiration passage within the needle body in an off-axis position. It is also expected that similar results will be obtained using a straight phacoemulsification needle having an aspiration passage that is formed with a cross-sectional configuration different than the cross-sectional configuration of the needle body itself, and that these results will be further amplified if the passage is also placed off-axis.

The inventors have herein determined that there is a need for further modification and improvement of phacoemulsification needles to enhance the safety of the phacoemulsification procedure. In some procedures, the needle distal end or tip may accidentally aspirate a portion of the posterior capsule during phacoemulsification, which can occlude the needle, applying significant vacuum to the delicate lens capsule tissues and risking a rupture thereof. The inventors have found that there is a need to prevent or at least minimize such occlusions.

The inventors have further determined that there is a need for modification and improvement of phacoemulsification needles to provide beneficial fluid management to prevent or at least minimize collapse or flattening of the anterior chamber, without the need of purchasing an expensive fluidics management system.

The inventors have further found that some interior surfaces of prior art needle tips may result in unwanted bounce-back or ejection of tissue particles from the opening of the aspiration passage or lumen in the needle body instead of being aspirated through the aspiration passage and transported through the needle body. Such bounce-back, repulsion, or surge decreases the efficiency of the overall aspiration of the needle and may increase the time of surgery.

While the following describes a preferred embodiment or embodiments of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as described and claimed herein

### SUMMARY OF THE INVENTION

In accordance with one preferred embodiment of the present invention, a phacoemulsification needle is provided for emulsifying body tissue. The needle is adapted to be attached to a phacoemulsification handpiece imparting a vibration to the needle. The needle has a hollow body having a distal end, a proximal end, and an internal surface defining an aspiration passage extending between the proximal and distal ends. The proximal end of the needle body is for mounting the needle body to a phacoemulsification handpiece. The needle body has a tip formed at its distal end. The aspiration passage defines a longitudinally-extending central body axis and the tip defines a longitudinally-extending central tip axis. The central tip axis is offset from the central body axis. The tip has at least one lateral aperture therein for enhanced safety in the event of occlusion of the tip during a phacoemulsification procedure.

In accordance with another preferred embodiment of the present invention a phacoemulsification needle is provided for emulsifying body tissue. The needle is adapted to be attached to a phacoemulsification handpiece imparting a vibration to the needle. The needle includes a needle body having a distal end, a proximal end, and an internal surface defining an aspiration passage extending between the proximal and distal ends. The proximal end is configured for mounting the needle body to a phacoemulsification handpiece. The needle body further includes an upper exterior surface and a lower exterior surface, and the needle body defines an internal lumen or aspiration passage having a longitudinally-extending central aspiration axis. The needle body defines a tip at its distal end having a longitudinally-extending central tip axis that is offset from the central aspiration axis. The tip has an upper tip surface that is coextensive with the upper exterior surface of the needle body, and the tip has a flaring, offset lower tip surface that extends radially outward from the needle body lower exterior surface. The upper tip surface has a first width and the offset lower tip surface having a second width, wherein the first width is greater than the second width.

In accordance with another preferred embodiment of the present invention a phacoemulsification needle is provided for emulsifying body tissue. The needle is adapted to be attached to a phacoemulsification handpiece imparting a vibration to the needle. The needle includes a needle body having a distal end, a proximal end, and an internal surface defining an aspiration passage extending between the proximal and distal ends. The proximal end is configured for mounting the needle body to a phacoemulsification handpiece. The needle body further includes an upper exterior surface and a lower exterior surface, and the needle body defines an internal lumen or aspiration passage having a longitudinally-extending central aspiration axis. The needle body defines a tip at its distal end having a longitudinally-extending central tip axis, the tip including at least a pair of opposing, concave, internal surfaces.

In accordance with another preferred embodiment of the present invention a phacoemulsification needle is provided for emulsifying body tissue. The needle is adapted to be attached to a phacoemulsification handpiece imparting a vibration to the needle. The needle includes a needle body having a distal end, a proximal end, and an internal surface defining an aspiration passage extending between the proximal and distal ends. The proximal end is configured for mounting the needle body to a phacoemulsification handpiece. The needle body further includes an upper exterior surface and a lower exterior surface, and the needle body defines an internal lumen or aspiration passage having a longitudinally-extending central aspiration axis. The needle body defines a tip at its distal end, the tip defining a longitudinally-extending central tip axis. A portion of the tip flares radially outwardly from the needle body with respect to the central body axis, the tip defining a first arcuate portion having a first radius of a given length, and the tip defining a second arcuate portion having a second radius of a given length. The second radius is substantially larger than the first radius. The second arcuate portion is located in the flared portion of the tip.

In accordance with another embodiment of the present invention a phacoemulsification needle is provided for emulsifying body tissue. The needle is adapted to be attached to a phacoemulsification handpiece imparting a vibration to the needle. The needle includes a needle body having a distal end, a proximal end, and an internal surface defining an aspiration passage extending between the proximal and distal ends. The proximal end is configured for mounting the needle body to a phacoemulsification handpiece. The needle body further includes an aspiration passage having a longitudinally-extending central aspiration axis. The needle body defines a tip at its distal end, the tip defining a longitudinally-extending central tip axis. The tip includes at least one internal projection therein.

It should be appreciated that the invention may include any of the detailed blockage reduction means described herein, either alone or in any combination. Furthermore, other objects, features and advantages of the invention will become apparent from a review of the entire specification including the appended claims and drawings.

### BRIEF DESCRIPTION OF THE DRAWINGS

FIG. 1 is an enlarged, side elevation view of a first embodiment of a phacoemulsification needle embodying the present invention;

FIG. 2 is a greatly enlarged fragmentary, cross-sectional view of just the tip portion of the needle illustrated in FIG. 1, taken in a plane extending through the center of the needle along the central body axis of the needle;

FIG. 2A is a greatly enlarged fragmentary, cross-sectional, detailed view of just the lip portion of the needle tip illustrated in FIG. 2,

FIG. 3 is a greatly enlarged, front elevation view of the needle tip illustrated in FIG. 1;

FIG. 4 is a greatly enlarged, fragmentary perspective view of the tip portion of the needle illustrated in FIG. 1;

FIG. 5 is a greatly enlarged, fragmentary, cross-sectional view of the tip portion of the needle of FIG. 2, and FIG. 5 illustrates the interior of the needle when the posterior capsular bag is inadvertently aspirated to occlude the tip of the needle and the lateral aspiration ports continue to permit aspiration of materials and fluid from the eye to decrease the vacuum or suction applied to the posterior capsular bag;

FIG. 6 is an enlarged, side elevation view of a second embodiment of a phacoemulsification needle according to the present invention;

FIG. 7 is a greatly enlarged, fragmentary perspective view of the tip portion of the needle illustrated in FIG. 6;

FIG. 8 is another a greatly enlarged, fragmentary perspective view of the tip portion of the needle illustrated in FIG. 6;

FIG. 9 is a greatly enlarged, front elevation view of the needle tip illustrated in FIG. 6;

FIG. 10 is a greatly enlarged, fragmentary, cross-sectional view of the tip portion of the needle illustrated in FIG. 6, taken in along plane 10-10 in FIG. 9;

FIG. 11 is a greatly enlarged fragmentary, cross-sectional view of the tip portion of the needle illustrated in FIG. 6, taken in along plane 11-11 in FIG. 9;

FIG. 12 is a greatly enlarged fragmentary, cross-sectional, perspective view of the tip portion of the needle illustrated in FIG. 6, taken in along plane 11-11 in FIG. 9;

FIG. 13 is an enlarged, front elevation view of a third embodiment of a phacoemulsification needle according to the present invention;

FIG. 14 is an enlarged fragmentary, cross-sectional view of the tip portion of the needle illustrated in FIG. 13, taken in along plane 14-14 in FIG. 13;

FIG. 15 is an enlarged, front elevation view of a fourth embodiment of a phacoemulsification needle according to the present invention;

FIG. 16 is an enlarged fragmentary, cross-sectional view of the tip portion of the needle illustrated in FIG. 15, taken in along plane 16-16 in FIG. 15;

FIG. 17 is an enlarged, side elevation view of a fifth embodiment of a phacoemulsification needle according to the present invention, and FIG. 17 shows just the tip and needle body portions of the needle;

FIG. 18 is an enlarged, front elevation view of the tip portion of the needle illustrated in FIG. 17;

FIG. 19 is an enlarged fragmentary, cross-sectional view of the tip portion of the needle illustrated in FIG. 17, taken in along plane 19-19 in FIG. 18;

FIG. 20 is an enlarged, side elevation view of a sixth embodiment of a phacoemulsification needle according to the present invention;

FIG. 21 is a greatly enlarged, front elevation view of the tip portion of the needle illustrated in FIG. 20;

FIG. 22 is a greatly enlarged fragmentary, cross-sectional view of the tip portion of the needle illustrated in FIG. 20, taken in along plane 22-22 in FIG. 21;

FIG. 23 is a greatly enlarged fragmentary, cross-sectional view of the tip portion of a seventh embodiment of a phacoemulsification needle according to the present invention, taken in along a plane extending through the center of the needle and dividing the needle into symmetric halves;

FIG. 24 is a greatly enlarged fragmentary, cross-sectional view of the tip portion of an eighth embodiment of a phacoemulsification needle according to the present invention, taken in along a plane extending through the center of the needle and dividing the needle into symmetric halves;

FIG. 25 is a greatly enlarged fragmentary, cross-sectional view of the tip portion of a ninth embodiment of a phacoemulsification needle according to the present invention, taken in along a plane extending through the center of the needle and dividing the needle into symmetric halves;

FIG. 26 is a greatly enlarged fragmentary, cross-sectional view of the tip portion of a tenth embodiment of a phacoemulsification needle according to the present invention, taken in along a plane extending through the center of the needle and dividing the needle into symmetric halves;

FIG. 27 is a greatly enlarged fragmentary, cross-sectional view of the tip portion of an eleventh embodiment of a phacoemulsification needle according to the present invention, taken in along a plane extending through the center of the needle and dividing the needle into symmetric halves; and

FIG. 28 is a greatly enlarged fragmentary, cross-sectional view of the tip portion of a twelfth embodiment of a phacoemulsification needle according to the present invention, taken in along a plane extending through the center of the needle and dividing the needle into symmetric halves.

### DETAILED DESCRIPTION OF THE PREFERRED EMBODIMENT

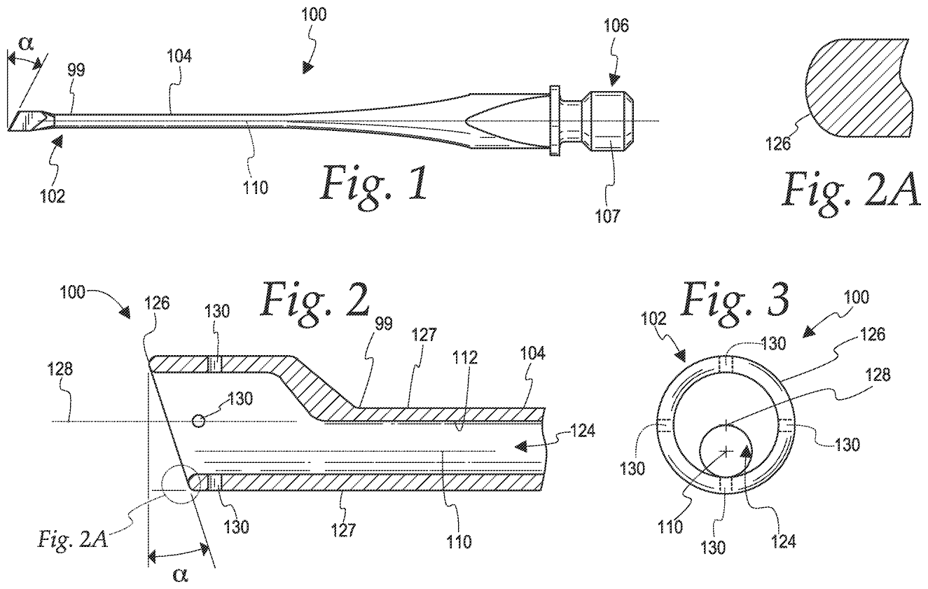

Referring now to FIG. 1, the numeral 100 indicates a first illustrated embodiment of a phacoemulsification needle embodying the present invention. The needle 100 is generally straight and has a needle body 104 that is preferably generally cylindrical and uniform along its length. It will be understood that the body 104 could be non-cylindrical or non-uniform in some applications. The body 104 terminates at an operative or distal end 99 and a proximal end 106, defining a length of the needle body 104. The needle distal end 99 has a tip 102, which will be discussed in greater detail hereinafter. The tip 102 preferably has a leading and trailing edge defined by an angle alpha that is about 30 degrees to a plane running perpendicular to the length of the needle body 104. The tip 102 need not be angled at all or may be defined by other angles. The needle proximal end 106 may have a mounting portion or hub 107 for connecting the needle 100 to a phacoemulsification handpiece (not shown). The needle 100 may be connected to the handpiece in any manner such as by mating male and female threads, clamping, snap fit, luer lock, friction fit, or other removable or adjustable type of connection.

Now referring to FIG. 2, the needle body 104 has an interior surface 112 defining an elongate lumen or aspiration passage 124 running from the proximal end 106 to the distal end 99 which also defines a central needle body axis 110. The central axis (geometric center) of the aspiration passage 124 is coincident with the needle body axis 110, however it will be understood that in some embodiments the central axis of the aspiration passage 124 may be offset from, or transverse to, the needle body axis 110 to introduce an advantage wobble to the needle during phacoemulsification. As described hereinafter, directions normally inward or normally outward from the needle body axis 110 are termed “radial” and directions along or parallel to the axis 110 are either toward the distal end 99 or the proximal end 106.

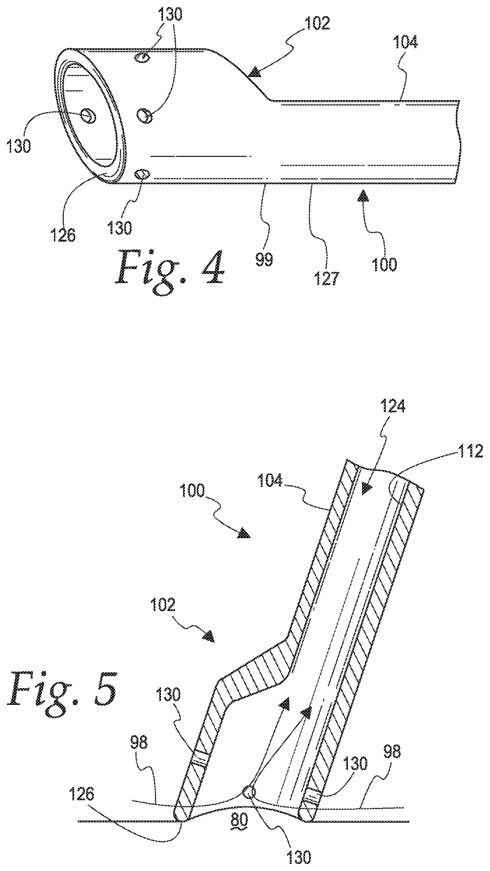

Referring now to FIGS. 2-4, which show a greatly enlarged view of the needle tip 102, the tip 102 can generally be characterized as having an offset or flaring shape in which the aspiration passage 124 opens up at the needle distal end 99 to a radially widened geometry when compared to the radial height or diameter of the aspiration passage 124 in the substantially cylindrical needle body 104 which is located proximally of the tip 102 along the central body axis 110. The tip 102 may be characterized as having an open or hollow mouth defined by, or terminating in, a circular perimeter or lip 126. As previously discussed, the tip 102 may have a leading edge and a trailing edge. The trailing edge is preferably contiguous or coincident with an outer surface 127 of the needle body 104, while the leading edge is radially offset, flaring away from the outer surface 127 of the needle body 104. However, in the broadest aspect of the invention, the tip 102 need not have any discernible leading or trailing edges, and the location of the leading and trailing edges may be positioned elsewhere along the lip 126.

Referring to FIGS. 2 and 3, the tip 102 defines a central tip axis 128 that is substantially parallel to, and offset from, the needle body central axis 110 by a distance 128. The aspiration passage 124 can be seen to connect to lip 126 via the open mouth. The first illustrated embodiment of the needle tip 102 can be seen to have a generally cylindrical shape terminating in a circular lip 126. The lip 126 is preferably rounded (FIG. 2A) or polished to enhance the safety of the needle 100 when contacting the lens capsule, particularly the posterior portions of the capsule. The inventors have found that the orientation of the central tip axis 128 to be offset from the body axis 110 can provide beneficial eccentric motion to the phacoemulsification needle distal end 99 during vibratory oscillation (longitudinal, torsional, or a blend thereof) by a handpiece.

As can be seen in FIG. 2, the needle body 104 outer surface 127 defines a first outer diameter and the 102 tip includes an outer surface defining a second outer diameter, the second outer diameter being between about 1.2 and 2 times larger than the first outer diameter, and more preferably, being about 1.5 times larger.

The material of the needle body 104 and tip 102 are preferably unitarily formed from a metal, but may be formed from a medical grade polymer, or a composite of metal and polymer. Preferably, at least the tip 102 and the lip 126 thereof have a mirror polish or surface finish for enhanced safety with respect to protecting delicate structures of the lens capsule.

Referring next to FIGS. 2, 3, and 4, the needle tip 102 includes at least one laterally extending aperture or irrigation bypass port 130. Preferably, the tip 102 includes a first pair of opposing lateral apertures 130 that extend in a first direction normal to the central body axis 110. In some applications, the tip 102 may be provided with a second pair of opposing lateral apertures 130 that extend in a second direction normal to the central body axis 110, the second pair of opposing lateral apertures 130 are spaced about ninety degrees from the first pair of opposing lateral apertures 130 (as can best be viewed in FIG. 3). In some applications (not illustrated), the apertures 130 may be angled or transverse relative to the central body axis 110 to direct flow toward the aspiration passage 124.

The apertures 130 are preferably manufactured in a secondary step of milling the needle tip 102. However, the apertures 130 may be created by other common manufacturing methods, such as being integrally formed in the metal needle 100, or removed by laser etching, electrical discharge machining, or other material removal operations.

The inventors have found that a phacoemulsification needle with a flaring, off-center tip, such as the tip 102, provides an ideal hold on the nucleus of the eye during phacoemulsification. It is believed that the wide mouth of the flaring tip 102 having a large surface area, which is followed by a relatively narrower surface area aspiration passage 124, contributes to this advantageous feature. Further, it has been found that this configuration having at least one lateral aperture 130 in the tip 102 increases the safety to the posterior lens capsule (e.g., 80 in FIG. 5) in the event that a portion of the posterior lens capsule is aspirated into the tip 102 to occlude the aspiration passage 124, whereby the at least one lateral aperture 130 perm its aspiration of particles and fluid (e.g., flow lines of aspirated materials designated as numeral 98) in the eye to minimize suction on the posterior lens capsule to prevent or at least minimize the potential for rupture of the lens capsule. It is believed that placement of the apertures 130 proximate to the lip 126 of the needle tip 102 is preferable for maintaining the efficiency of the needle 100 during operation.

In the presently preferred embodiment of the needle 100, the lateral apertures 130 are circular and define a diameter of about 0.1 mm. The diameter of the circular aspiration passage 124 is between 2 and 10 times larger than the diameter of the lateral apertures 130, and are more preferably about 5 times larger than the diameter of the lateral apertures 130.

It will be understood that in one broad form of the present invention, the needle 100 could be provided with one or more lateral apertures 130 in a tip 102 that is offset, but not flaring, or may be provided in a tip that is even not flaring nor offset in some applications.

The inventive needle 100, which has a means for decreasing the risk of posterior capsule rupture as described above, may advantageously be used with a variety of vibratory handpieces which can impart a longitudinal, torsional, elliptical, and/or blended vibrations to the needle. Furthermore, such an improved needle may eliminate the need for employing an expensive fluidics management system when performing phacoemulsification on tissues of the eye.

The inventive needle 100, which has a means for decreasing the risk of posterior capsule rupture as described above, may advantageously be used with a needle that is not generally straight, and that is bent, stepped, or angled along its length (not illustrated).

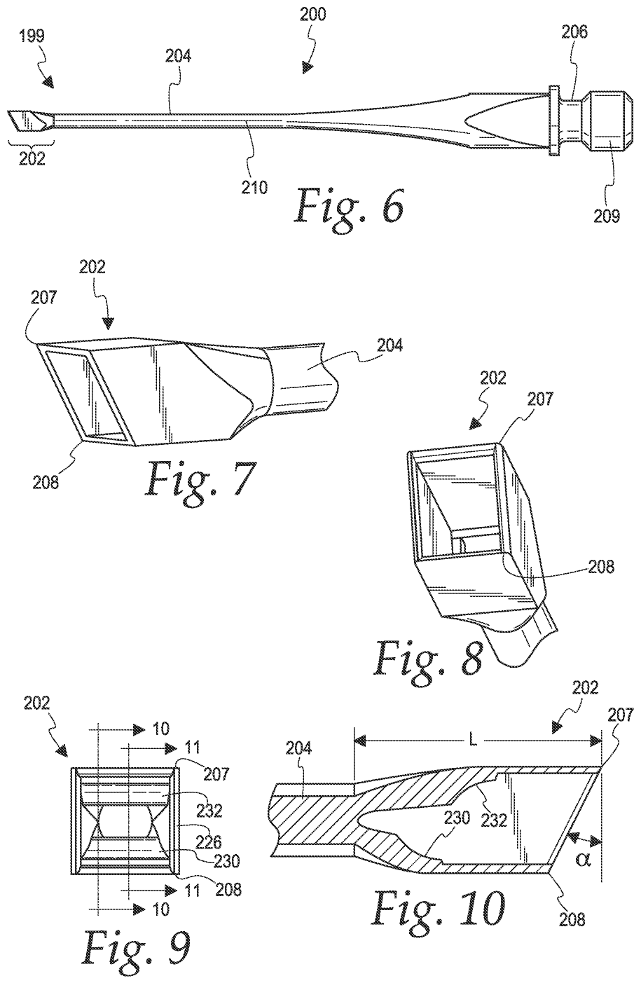

Referring now to FIGS. 6-12, the numeral 200 indicates a second embodiment of a phacoemulsification needle embodying the present invention. The needle 200 is generally straight and has a needle body 204. The body has an operative or distal end 199 and a proximal end 206, defining a length of the needle body 204. A tip 202 extends from the needle distal end 199. The tip 202 has a leading edge 207 and trailing edge 208 defined by an angle a that is about 30 degrees to the plane running perpendicular to the length of the needle body 204. The tip 202 need not be angled at all or may be defined by other angles. The needle proximal end 206 has a mounting portion or hub 209 for connecting the needle 200 to a phacoemulsification vibratory handpiece (not illustrated). The needle 200 may be connected to the handpiece in any manner such as by mating threads, clamping, snap fit, lock, friction fit, or other similar removable connection.

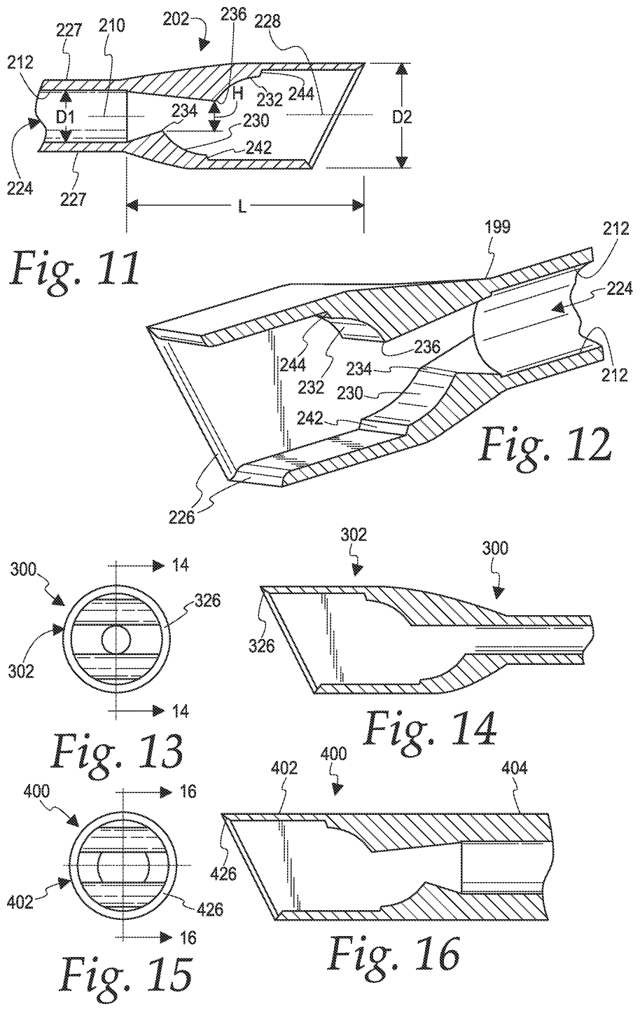

Now referring to FIG. 11, the needle body 104 has an interior surface 212 defining an elongate lumen or aspiration passage 224 running from the proximal end 206 to the distal end 199 and defining a central needle body axis 210. As described hereinafter, directions inward or outward from the needle body axis 210 are termed “radial”, and “axial” directions along the axis 210 are either toward the distal end 199 or the proximal end 206.

Referring now to FIGS. 11 and 12, which each show a greatly enlarged view of the needle tip 202, the tip 202 can generally be characterized as having an offset or flaring shape extending axially away from the aspiration passage 224 such that the tip 202 is radially widened at the needle body distal end 199 when compared to the diameter D1 (FIG. 11) of the aspiration passage 224 in the substantially cylindrical needle body 204 which is located proximally of the tip 202 along the axis 210. The tip 202 may be characterized as having an open mouth terminating in a generally square lip 226. As previously discussed, the tip 202 has a leading edge 207 and a trailing edge 208. Each of the leading and trailing edges 207/208 is radially or laterally offset from an outer surface 227 of the needle body 204.

Referring to FIG. 11, the tip 202 defines a central tip axis 228 that is substantially parallel to, and coincident with, the needle body central axis 210. The aspiration passage 224 can be seen to connect to lip 226 via the open mouth of the tip 202.

The needle tip 202 can be seen to have a generally square shape terminating in a square lip 226 (FIGS. 9 and 12). The lip 226 is preferably rounded to enhance the safety of the needle 200 when contacting the lens capsule, particularly the posterior portions of the capsule.

Referring now to FIGS. 9, 11, and 12, the tip 202 further defines at a pair of opposing, concave, internal surfaces 230/232. The tip 202 defines a length “L” in the direction along the longitudinally-extending central tip axis 228, and the pair of opposing, concave, internal surfaces 230/232 extend along (begin and end at) different locations of the length “L” of the tip 202. In other words, the concave, internal surfaces 230/232 are not symmetric about a horizontal plane extending through the central tip axis 228, and more preferably are not overlapping (i.e., one of the surfaces 230 does not overlie the other surface 232) along the length “L”, as can best be seen in FIG. 11. Each one of the pair of opposing, concave, internal surfaces 230/232 defines a different radius of curvature, whereby the surface 230 defines a larger radius of curvature than the opposing surface 232.

Referring next to FIGS. 11 and 12, each one of the pair of opposing, concave, internal surfaces 230/232 connects at a radially innermost point 234/236 to a sloping or ramped surface 238/240 that extends to the needle body 204 internal surface 212 defining the aspiration passage 224. The radially innermost points 234/236 are separated by a restriction or normal height “H” (FIG. 11) in a radial direction relative to the longitudinally-extending central body axis 228. The height “H” is substantially less than the diameter D1 (FIG. 11) of the aspiration passage 124 in the needle body 202, preferably between about 20-50% less. Each one of the pair of opposing, concave, internal surfaces includes a radially-offset step 242/244 located at its leading edge.

The inventors have found that the needle having a tip with a pair of opposing, concave, internal surfaces may provide enhanced efficiency of phacoemulsification by the provision of asymmetric, internal cutting edges for mulching or degrading the nucleus material to prevent or at least minimize clogging of the aspiration passage. In addition, the restriction in the tip is believed to function to accelerate aspirated lens tissues to create a multiplier effect for the phacoemulsification system vacuum. It is currently believed that the tip 202 will require less energy usages during ultrasound operation of the handpiece as compared to prior art commercially-available needles. While the needle 200 is suitable for a multitude of handpieces, such as longitudinal, torsional, blended handpieces, it is believed that the internal surface structures are most suited for, and effective with, a longitudinally-vibrating handpiece.

The material of the needle body 200 and tip 202 are preferably unitarily formed from a metal (e.g., steel, titanium, or alloy), but may be formed from a medical grade polymer, or composite of metal and polymer. Preferably, at least the tip 202 and the lip 226 thereof have a mirror polish or surface finish for enhanced safety with respect to protecting delicate structures of the lens capsule. Some surfaces of the tip 202 may have a sandblasted or roughened finish to facilitate polishing of the lens capsule. A portion, or all of, the tip 202 may also have an overmolded polymer formed thereon (thermoplastic, elastomer, silicone, etc.) for enhanced safety.

FIGS. 13-14 illustrate a third embodiment of a needle according to the present invention designated by the numeral 300. The needle 300 operates in a similar manner as the second embodiment 200 described above, with analogous advantageous internal, concave surface features. However, the third embodiment of the phacoemulsification needle 300 differs in defining a tip 302 having a generally circular shape terminating in a circular lip 326.

FIGS. 15-16 illustrate a fourth embodiment of a needle according to the present invention designated by the numeral 400. The needle 400 operates in a similar manner as the second embodiment 200 described above, with analogous advantageous internal, concave surface features. However, the fourth embodiment of the phacoemulsification needle 400 differs in that it defines a needle body 404 that is coincident with a non-flaring tip 402, both having a generally circular shape of the same outer diameter, and terminating in a circular lip 426.

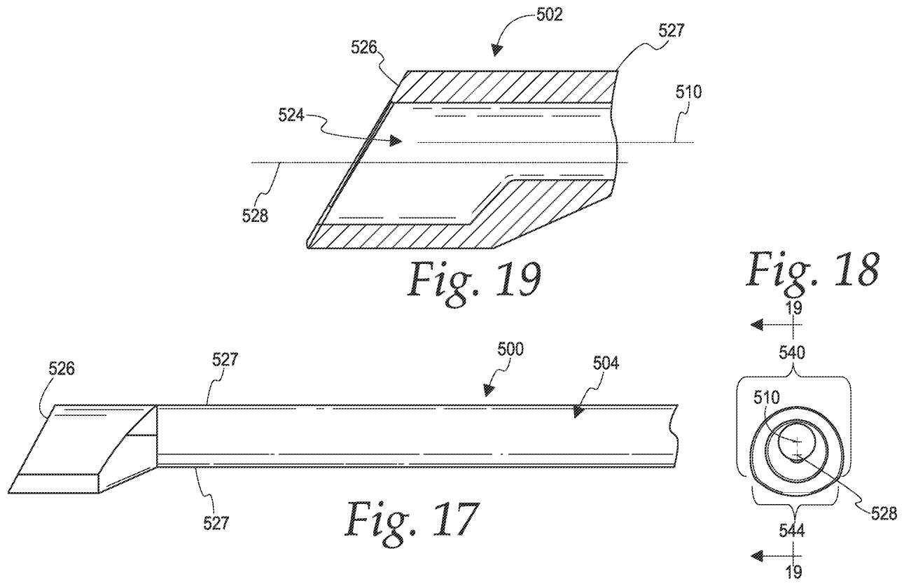

FIGS. 17-19 illustrate a fifth embodiment of a needle according to the present invention designated by the numeral 500. The needle 500 operates in a similar manner as the first embodiment of the needle 100 described above. As with that prior discussed embodiment, the needle 500 also includes a distal tip 502, a generally cylindrical needle body 504 defining a central body axis 510, a hollow aspiration passage 524 extending therein, and a proximal end (not illustrated but identical to the prior discussed embodiments) for being affixed to a vibratory handpiece. The body 504 defines a cylindrical outer surface 527 from which a lower portion of the tip 502 radially extends or flares outwardly. The tip 502 defines a central tip axis 528 that is substantially parallel to, and offset from, the central body axis 510.

However, the fifth embodiment of the phacoemulsification needle 500 differs from the prior-discussed embodiment of the needle 100 in that the tip 502, when viewed in a plane normal to the axis 510 as in FIG. 18, defines a first arcuate portion 540 having a first radius of curvature, and a second arcuate portion 544 having a second radius curvature, wherein the second radius is larger than the first radius. Furthermore, it can be seen that the second arcuate portion 544 is located within the flared portion of the tip 502. The flared portion of the tip 502 has a decreased wall thickness compared to the non-flared portion (portion that is coextensive with the needle body outer surface 527).

The inventors believe that the needle 500 having a tip 502 will allow for a smoother incision entry and add extra protection to the posterior capsule when compared to prior art needles. The inventors further believe that the tip 502 may provide a more enhanced wobble effect to improve the efficiency of phacoemulsification. The needle 500 may be suitable for a multitude of handpieces, such as longitudinal, torsional, blended handpieces.

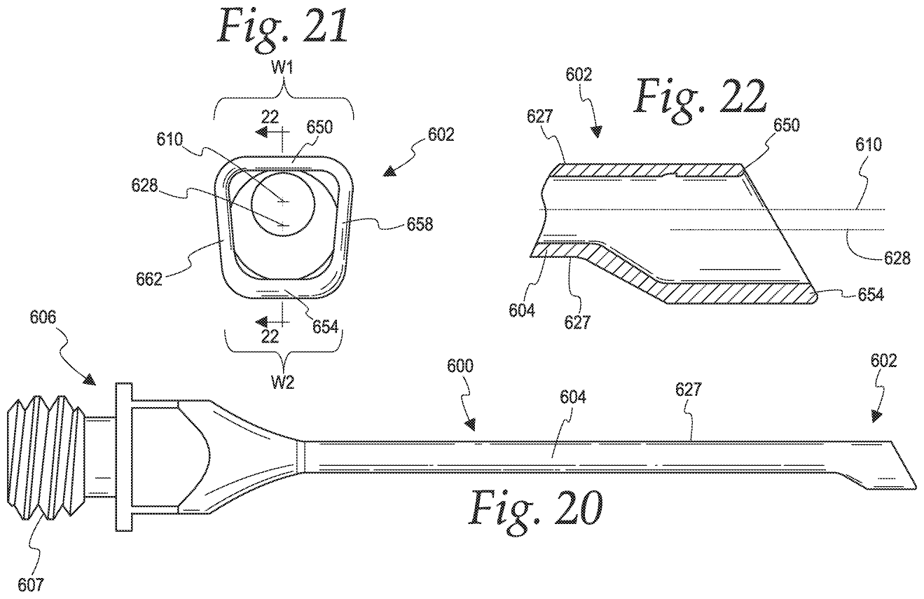

FIGS. 20-22 illustrate a sixth embodiment of a needle according to the present invention designated by the numeral 600. The needle 600 operates in a similar manner as the first embodiment of the needle 100 described above. The needle 600 also includes a distal tip 602, a generally cylindrical needle body 604 defining a central body axis 610, a hollow aspiration passage 624 extending therein, and a proximal end 606. The needle proximal end 606 includes a mounting portion or hub 607 for connecting the needle 600 to a phacoemulsification handpiece (not shown), as with the prior embodiments discussed above, in this case shown as male screw threads. The body 604 defines an outer surface 627 from which a lower portion of the tip 602 radially extends or flares outwardly therefrom. The tip 602 defines a central tip axis 628 that is substantially parallel to, and offset from, the central body axis 610.

With reference to FIG. 21, the sixth illustrated embodiment of the phacoemulsification needle 600 differs from the prior-discussed embodiments in that the tip 602 defines a generally trapezoidal or inverted trapezoid shape when viewed in a plane extending normal to the tip axis 628. Specifically, the tip 602 includes an upper tip surface or wall 650 that is coextensive with the upper exterior surface 627 of the needle body 604. The tip 602 further includes an opposite, offset lower tip surface or wall 654 that extends radially outward of the needle body lower exterior surface 627. The tip 602 further includes a pair of lateral side surfaces or walls 658/662 which slope inwardly toward the central tip axis 628 from the upper tip surface 650 to the lower tip surface 654.

Still referring to FIG. 21, the upper tip wall 650 defines a first width W1 that is greater than a second width W2 defined by the offset lower tip wall 654. The first width W1 of the upper tip wall 650 is preferably between 5 and 15 percent larger than the second width W2 of the offset lower tip wall 654, and more preferably the first width W1 of the upper tip wall 650 is between 9 and 11 percent larger than the second width W2 of the offset lower tip wall 654.

Still referring to FIG. 22, the bottom tip wall 654 extends axially outwardly (distally) of upper tip wall 650 along the central tip axis 628. In addition, bottom tip wall 654 is at least 30 percent thicker than upper tip surface 650.

The inventors believe that the needle 600 having a tip 602 with the somewhat trapezoidal configuration will also allow for a smoother incision entry and add extra protection to the posterior capsule compared to the prior art square tips. The inventors further believe that the tip 602 may provide a more enhanced wobble effect to improve the efficiency of phacoemulsification compared to prior art needles in general. The needle 600 may be suitable for a multitude of handpieces, such as longitudinal, torsional, blended handpieces.

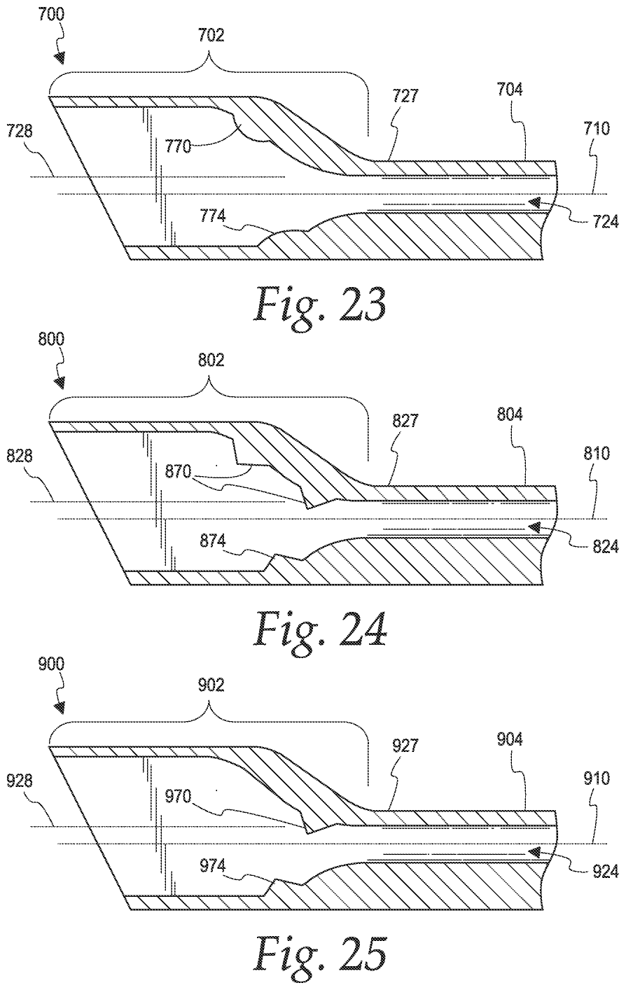

FIGS. 23-28 illustrate additional embodiments of phacoemulsification needles according to the present invention containing internal elements or projections within the tip portion of the needle for improving the efficiency of the needle and minimization of clogging during a phacoemulsification operation. These needles operate in a similar manner as the first embodiment of the needle 100 described above, and only the tip portion of the needles are illustrated. It will be understood that the remainder of the proximal end and body of these additional needle is consistent with the structure shown in FIG. 1 or described above. Furthermore, the needle tips of these embodiments may be generally square, circular, flaring, or non-flaring, as shown and described above. The inventors believe that these additional needle embodiments will allow for a more efficient phacoemulsification (reduction of energy used) and a reduced potential for clogging of phacoemulsification compared to prior art needles in general. While these needles may be suitable for a multitude of handpieces, such as longitudinal, torsional, blended handpieces, it is presently believed that the needles are most suited for longitudinally-moving handpieces, such as a longitudinally-vibrating piezoelectric handpiece or a longitudinally-moving pressure type handpiece (such as is shown in United States Patent Application Publication US20180318132 A1, which is incorporated by reference herein in its entirety).

Referring now to FIG. 23, a seventh embodiment of a needle according to the present invention is designated by the numeral 700 and includes a distal tip 702, a generally cylindrical needle body 704 defining a central body axis 710, a hollow aspiration passage 724 extending therein, and a proximal end for being attached to a vibratory handpiece, as with the prior embodiments discussed above. The body 704 defines an outer surface 727 from which a portion of the tip 702 radially extends or flares outwardly therefrom. The tip 702 defines a central tip axis 728 that is substantially parallel to, and offset from, the central body axis 710. The phacoemulsification needle 700 differs from the prior-discussed embodiments in that the tip 702 defines a first internal mulching projection 770 in the form of a convex protrusion along the offset or flared portion of the tip 702 and a second internal mulching projection 774 in the form of a convex protrusion along the non-flared, coextensive portion of the tip 702. The mulching projections 770, 774 are axially offset from one another, along the central body axis 710 and lead into the aspiration passage 724. The mulching projections 770, 774 may extend as a line or wall out of the plane of view in FIG. 23, or they may be a plurality of spaced-apart, discrete projections that extend generally as a line out of the plane of view in FIG. 23

Referring now to FIG. 24, an eighth embodiment of a needle according to the present invention is designated by the numeral 800 and includes a distal tip 802, a generally cylindrical needle body 804 defining a central body axis 810, a hollow aspiration passage 824 extending therein, and a proximal end for being attached to a vibratory handpiece, as with the prior embodiments discussed above. The body 804 defines an outer surface 827 from which a portion of the tip 802 radially extends or flares outwardly therefrom. The tip 802 defines a central tip axis 828 that is substantially parallel to, and offset from, the central body axis 810. The phacoemulsification needle 800 differs from the prior-discussed embodiments in that the tip 802 defines first and second internal mulching projections 870 in the form of a pointed, triangular protrusion along the offset or flared portion of the tip 802 and a third internal mulching projection 874 in the form of a pointed, triangular protrusion along the non-flared, coextensive portion of the tip 802. The mulching projections 870, 874 are axially offset from one another, along the central body axis 810 and lead into the aspiration passage 824. The mulching projections 870, 874 may extend as a line or wall out of the plane of view in FIG. 24, or they may be a plurality of spaced-apart, discrete projections that extend as a line out of the plane of view in FIG. 24.

Referring now to FIG. 25, a ninth embodiment of a needle according to the present invention is designated by the numeral 900 and includes a distal tip 902, a generally cylindrical needle body 904 defining a central body axis 910, a hollow aspiration passage 924 extending therein, and a proximal end for being attached to a vibratory handpiece, as with the prior embodiments discussed above. The body 904 defines an outer surface 927 from which a portion of the tip 902 radially extends or flares outwardly therefrom. The tip 902 defines a central tip axis 928 that is substantially parallel to, and offset from, the central body axis 910. The phacoemulsification needle 900 differs from the prior-discussed embodiments in that the tip 902 defines first internal mulching projection 970 in the form of a pointed, triangular protrusion along the offset or flared portion of the tip 902 and a second internal mulching projection 974 in the form of a pointed, triangular protrusion along the non-flared, coextensive portion of the tip 902. The mulching projections 970, 974 are axially offset from one another, along the central body axis 910 and lead into the aspiration passage 924. The mulching projections 970, 974 may extend as a line or wall out of the plane of view in FIG. 25, or they may be a plurality of spaced-apart, discrete projections that extend as a line out of the plane of view in FIG. 25.

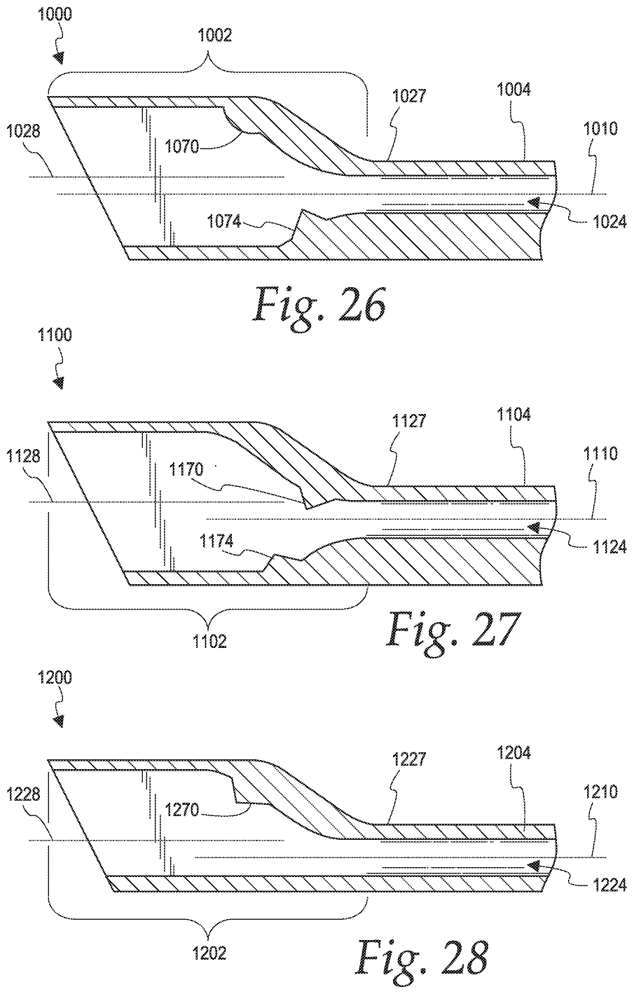

Referring now to FIG. 26, a tenth embodiment of a needle according to the present invention is designated by the numeral 1000 and includes a distal tip 1002, a generally cylindrical needle body 1004 defining a central body axis 1010, a hollow aspiration passage extending therein, and a proximal end for being attached to a vibratory handpiece, as with the prior embodiments discussed above. The body 1004 defines an outer surface 1027 from which a portion of the tip 1002 radially extends or flares outwardly therefrom. The tip 1002 defines a central tip axis 1028 that is substantially parallel to, and offset from, the central body axis 1010. The phacoemulsification needle 1000 differs from the prior-discussed embodiments in that the tip 1002 defines first internal mulching projection 1070 in the form of a convex protrusion extending along the offset or flared portion of the tip 1002 and a second internal mulching projection 1074 in the form of a pointed, triangular protrusion along the non-flared, coextensive portion of the tip 1002. The mulching projections 1070, 1074 are axially offset from one another, along the central body axis 1010 and lead into the aspiration passage. The mulching projections 1070, 1074 may extend as a line or wall out of the plane of view in FIG. 26, or they may be a plurality of spaced-apart, discrete projections that extend as a line out of the plane of view in FIG. 26.

Referring now to FIG. 27, an eleventh embodiment of a needle according to the present invention is designated by the numeral 1100 and includes a distal tip 1102, a generally cylindrical needle body 1104 defining a central body axis 1110, a hollow aspiration passage extending therein, and a proximal end for being attached to a vibratory handpiece, as with the prior embodiments discussed above. The body 1104 defines an outer surface 1127 from which a portion of the tip 1102 radially extends or flares outwardly therefrom. The tip 1102 defines a central tip axis 1128 that is substantially parallel to, and offset from, the central body axis 1110. The phacoemulsification needle 1100 differs from the prior-discussed embodiments in that the tip 1102 defines first internal mulching projection 1170 in the form of a steeply pointed, triangular protrusion extending along the offset or flared portion of the tip 1102 in a direction essentially normal to the axis 1128 of the tip 1102 and a second internal mulching projection 1174 in the form of a steeply pointed, triangular protrusion along the non-flared, coextensive portion of the tip 1102 in a direction essentially normal to the axis 1128 of the tip 1102. The mulching projections 1170, 1174 are axially offset from one another, along the central body axis 1110 and lead into the aspiration passage. The mulching projections 1170, 1174 may extend as a line or wall out of the plane of view in FIG. 27, or they may be a plurality of spaced-apart, discrete projections that extend as a line out of the plane of view in FIG. 27.

Referring now to FIG. 28, a twelfth embodiment of a needle according to the present invention is designated by the numeral 1200 and includes a distal tip 1202, a generally cylindrical needle body 1204 defining a central body axis 1210, a hollow aspiration passage extending therein, and a proximal end for being attached to a vibratory handpiece, as with the prior embodiments discussed above. The body 1204 defines an outer surface 1227 from which a portion of the tip 1202 radially extends or flares outwardly therefrom. The tip 1202 defines a central tip axis 1228 that is substantially parallel to, and offset from, the central body axis 1210. The phacoemulsification needle 1200 differs from the prior-discussed embodiments in that the tip 1202 defines first internal mulching projection 1270 in the form of a steeply pointed, triangular protrusion extending along the offset or flared portion of the tip 1202 in a direction essentially normal to the axis 1228 of the tip 1202. The mulching projection 1270 may extend as a line or wall out of the plane of view in FIG. 28, or it may be a plurality of spaced-apart, discrete projections that extend as a line out of the plane of view in FIG. 28.

The foregoing disclosure of specific embodiments is intended to be illustrative of the broad concepts comprehended by the invention.

1.-33. (canceled)

34. A phacoemulsification needle for emulsifying body tissue, said needle adapted to be attached to a phacoemulsification handpiece for imparting a vibration to said needle, said needle comprising:
a hollow needle body having a distal end, a proximal end, and an internal surface defining an aspiration passage extending between said proximal and distal ends, said proximal end for mounting said needle body to a phacoemulsification handpiece,
said aspiration passage defining a longitudinally-extending central body axis,
said needle body defining a tip at said distal end, said tip defining a longitudinally-extending central tip axis, wherein a portion of said tip flares radially outwardly from said needle body with respect to said central body axis, said tip defining a first arcuate portion having a first radius, and said tip defining a second arcuate portion having a second radius, said second radius being larger than said first radius, and said second arcuate portion located in said flared portion of said tip.

35. The phacoemulsification needle of claim 34 wherein said flared portion of said tip has a decreased wall thickness compared to a non-flared portion that is coextensive with a needle body outer surface.

36. The phacoemulsification needle of claim 34 wherein said tip includes at least one lateral aperture therein.

37. The phacoemulsification needle of claim 36 wherein said at least one lateral aperture in said tip has the form of a first pair of opposing lateral apertures and a second pair of opposing lateral apertures oriented 90 degrees from said first pair of opposing lateral apertures relative to said central tip axis.

38. The phacoemulsification needle of claim 34 wherein said needle body includes a generally cylindrical outer surface and said tip includes a generally cylindrical outer surface.

39. The phacoemulsification needle of claim 34 wherein said tip terminates in a lip, said lip defining a radiused distal surface.

40. The phacoemulsification needle of claim 34 wherein said tip has at least one internal projection therein.

41. The phacoemulsification needle of claim 40 wherein said at least one internal projection has the form of a pair of longitudinally-offset arcuate, convex surfaces when viewed in a cross-sectional plane extending through said central tip axis.

42. The phacoemulsification needle of claim 40 wherein said at least one internal projection has the form of a pair of longitudinally-offset substantially triangular surfaces when viewed in a cross-sectional plane extending through said central tip axis.

42. The phacoemulsification needle of claim 40 wherein said at least one internal projection extends within said flared portion of said tip.

43. The phacoemulsification needle of claim 34 wherein said tip defines at least a pair of opposing, concave, internal surfaces.

44. The phacoemulsification needle of claim 43 said pair of opposing, concave, internal surfaces extend along different locations of said length of said tip.

45. The phacoemulsification needle of claim 43 wherein one of said pair of opposing, concave, internal surfaces extends within said flared portion of said tip.

46. The phacoemulsification needle of claim 43 wherein said tip defines a length along said longitudinally-extending central tip axis, and wherein said pair of opposing, concave, internal surfaces do not overlap along said length.

47. The phacoemulsification needle of claim 43 wherein each one of said pair of opposing, concave, internal surfaces defines a different radius of curvature.

48. The phacoemulsification needle of claim 43 wherein each one of said pair of opposing, concave, internal surfaces connects at a radially innermost point to a ramped surface that extends to said internal surface defining said aspiration passage.

49. The phacoemulsification needle of claim 48 wherein said radially innermost points are separated by a height in a radial direction relative to said longitudinally-extending central body axis, and said height is less than a diameter of said aspiration passage in said radial direction relative to said longitudinally-extending central body axis.

50. The phacoemulsification needle of claim 43 wherein each one of said pair of opposing, concave, internal surfaces includes a leading edge and a trailing edge, and each leading edge connects to a radially-offset step in said tip.

51. The phacoemulsification needle of claim 34 in combination with a vibratory handpiece.
